(19) United States
(12) Patent Application Publication
(10) Pub. No.: US 2009/0312297 A1
(43) Pub. Date: Dec. 17, 2009

(54) METHODS FOR TREATING HYPERCHOLESTEROLEMIA AND ATHEROSCLEROSIS

(75) Inventors: Gokhan S. Hotamisligil, Wellesley, MA (US); Umut Ozcan, Brookline, MA (US)

Correspondence Address: EDWARDS ANGELL PALMER & DODGE LLP, P.O. BOX 55874, BOSTON, MA, 02205, US

(73) Assignee: PRESIDENT AND FELLOWS OF HARVARD COLLEGE, Cambridge, MA (US)

(21) Appl. No.: 12/293,970

(22) PCT Filed: Mar. 22, 2007
(86) PCT No.: PCT/US2007/007225

§ 371 Date: Apr. 24, 2009

Related U.S. Application Data
(60) Provisional application No. 60/785,156, filed on Mar. 22, 2006.

(51) Int. Cl. A61K 31/567; A61K 31/19; A61K 31/13; A61P 9/10

(52) U.S. Cl. 514/182; 514/557; 514/644

(57) ABSTRACT

The invention provides compounds and pharmaceutical compositions that can be used to treat or prevent atherosclerosis, stroke, and other ischemic vascular diseases, dyslipidemia and hypercholestcrolemia and prevent complications of these conditions. Agents in accordance with the invention include; tauroursodeoxycholic acid (TUDCA), and analogs and derivatives thereof; 4-phenyl butyric acid (PBA), and analogs and derivatives thereof; and trimethyl N-oxide (TMAO), and analogs and derivatives thereof.

### RELATED APPLICATIONS

This application claims priority to U.S. Provisional Patent application 60/785,156, filed Mar. 22, 2006, which has the same title as the instant application. This application is also related to the subject matter of U.S. patent application Ser. No. 11/227,497, filed Sep. 15, 2005, and U.S. patent application Ser. No. 11/227,543, filed Sep. 15, 2005, the contents of all of which are incorporated herein by reference.

### GOVERNMENT SUPPORT

The work described herein was supported, in part, by a grant from the National Institutes of Health. The United States government may have certain rights in the invention.

### BACKGROUND OF THE INVENTION

Hypercholesterolemia is a prevalent and growing health problem throughout the world. Hypercholesterolemia refers to the presence of high or excessive levels of cholesterol in the blood. Hypercholesterolemia can lead to the development of atherosclerotic plaques in arteries and, eventually, to atherosclerosis, stroke, ischemic vascular disease, dyslipidemia and hypercholesterolemia and other complications of these conditions. These cholesterol-associated diseases have become serious threats to human health.

Hypercholesterolemia may be associated with the activation of cellular stress signaling pathways. One player in the cellular stress response is the endoplasmic reticuluin (ER), a membranous network that functions in the synthesis and processing of secretory and membrane proteins. The ER is responsible for the processing and translocation of most secreted and integral membrane proteins of eukaryotic cells. The lumen of the ER provides a specialized environment for the posttranslational modification and folding of these proteins. Properly folded proteins are cleared for exit from the ER and progress down the secretory pathway, while unfolded or misfolded proteins are disposed of by ER-associated protein degradation machinery. The load of proteins that cells process varies considerably depending on the cell type and physiological state of the cell. Cells can adapt by modulating the capacity of their ER to process proteins and the load of protein synthesized. Disequilibrium between ER load and folding capacity is referred to as ER stress (Harding et al. Diabetes 51(Supp. 3):S455, 2002). ER stress has been shown to be triggered by hypoxia, hypoglycemia, exposure to natural toxins that perturb ER function, and a variety of mutations that affect the ability of client proteins to fold (Lee, Trends Biochem. Sci. 26:504-510, 2001; Lee, Curr. Opin. Cell Biol. 4:267-273, 1992).

Certain pathological conditions have been shown to disrupt ER homeostasis thereby leading to the accumulation of unfolded and misfolded proteins in the ER lumen (Hampton Curr. Biol. 10:R518, 2000; Mori Cell 101:451, 2000; Harding et al. Annu. Rev. Cell Dev. Biol. 18:575, 2002). To cope with ER stress, cells activate a signal transduction system linking the ER lumen with the cytoplasm and nucleus, called the unfolded protein response (UPR) (Hampton Curr. Biol 10:R518, 2000; Mori Cell 101:451, 2000; Harding et al. Annu. Rev. Cell Dev. Biol. 18:575, 2002). Among the conditions that trigger ER stress are glucose and nutrient deprivation, viral infections, increased synthesis of secretory proteins, and the expression of mutant or misfolded proteins (Ma et al. Cell 107:827,2001; Kaufman et al. Nat. Rev. Mol. Cell Biol. 3:411, 2002).

### SUMMARY OF THE INVENTION

The inventors have discovered that endoplasmic reticulum stress is a key link between obesity, insulin resistance, and type 2 diabetes (Ozcan et al., Science 313:1137-1140, 2006). Insulin resistance is a common feature of obesity and predisposes individuals to a variety of pathologies, including hypertension, dyslipidemias, cardiovascular disease, and type 2 diabetes mellitus. Moreover, obesity and atherosclerosis have both been associated with inflammation. Ozcan et al. (2006) demonstrated that reduction of ER stress by administration of chemical chaperones restore glucose homeostasis in a mouse model of type 2 diabetes and enhance insulin sensitivity in liver, muscle, and adipose tissue.

The instant invention is based, at least in part, on the discovery that reducing ER stress can be to treat hypercholesterolemia and atherosclerosis. Thus, in one aspect, the invention provides methods for treating or preventing hypercholesterolemia and/or atherosclerosis in a subject comprising administering to a subject in need thereof a therapeutically effective amount of a TUDCA compound of formula I:

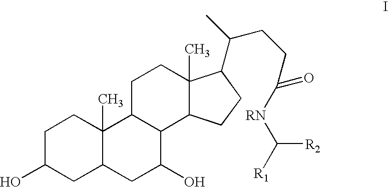

wherein R is —H or C₁-C₄ alkyl;

R₁ is —CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and

R₃ is —H or the residue of a basic amino acid, or

a pharmaceutically acceptable salt or derivative thereof; or a mixture thereof, thereby treating or preventing hypercholesterolemia and/or atherosclerosis in said subject.

In another aspect, the invention provides methods for treating or preventing hypercholesterolemia and/or atherosclerosis in a subject comprising administering to a subject in need thereof a therapeutically effective amount of a PRA compound of formula II:

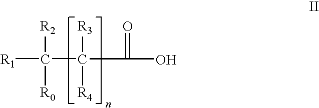

wherein n is 1 or 2;

R₀ is aryl, heteroaryl, or phenoxy, the aryl and phenoxy being unsubstituted or substituted with, independently, one or more halogen, hydroxy or lower alkyl;

R₁ and R₂ are independently H, lower alkoxy, hydroxy, lower alkyl or halogen; and

a pharmaceutically-acceptable salt thereof; or a mixture thereof, thereby treating or preventing hypercholesterolemia and/or atherosclerosis in said subject.

In another aspect, the invention provides methods for treating or preventing hypercholesterolemia and/or atherosclerosis in a subject comprising administering to a subject in need thereof a therapeutically effective amount of a TMAO compound of formula III:

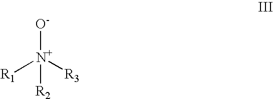

wherein

R₁, R₂, and R₃ are independently hydrogen, halogen, or lower C₁-C₆ alkyl; or

a pharmaceutically-acceptable salt thereof; or a mixture thereof, thereby treating or preventing hypercholesterolemia and/or atherosclerosis in said subject.

In an aspect, the invention provides the use of the TUDCA, PBA, and TMAO compounds of the invention for use in the preparation of a medicament for treatment or prevention of atherosclerosis or hypercholesterolemia.

In an aspect of the invention, pharmaceutical compositions and medicaments of the invention include the TUDCA, PBA, and TMAO compounds used in the invention and pharmaceutically acceptable excipients are also provided. The pharmaceutical compositions may be formulated for oral, parenteral, or transdermal delivery. The compound of the invention may also be combined with other pharmaceutical agents.

In an aspect, the invention provides kits that include the TUDCA, PBA, and TMAO compounds used in the invention. The kit may also include instructions for the physician and/or patient, syringes, needles, box, bottles, vials, etc.

In an aspect, the invention provides methods and agents that are useful in preventing or treating hypercholesterolemia, atherosclerosis and associated diseases. In particular, the invention provides agents or pharmaceutical compositions that can be used to treat or prevent atherosclerosis, stroke, and other ischemic vascular diseases, dyslipidemia and hypercholesterolemia and prevent complications of these conditions.

The invention provides for the use of the compositions of the invention for the preparation of a medicament for preventing or treating hypercholesterolemia, atherosclerosis and associated diseases.

### Definitions

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). In certain embodiments, the animal is a human.

“Chemical chaperone”: A “chemical chaperone” is a compound known to stabilize protein conformation against denaturation (e.g., chemical denaturation, thermal denaturation), thereby preserving protein structure and function (Welch et al. Cell Stress Chaperones 1:109-115, 1996). In certain embodiments, the “chemical chaperone” is a small molecule or low molecular weight compound. Preferably, the “chemical chaperone” is not a protein. Examples of “chemical chaperones include glycerol, deuterated water (D₂O), dimethylsulfoxide (DMSO), trimethylamine N-oxide (TMAO), glycine betaine (betaine), glycerolphosphocholine (GPC) (Burg et al. Am. J. Physiol. (Renal Physiol. 43):F762-F765, 1998), 4-phenyl butyrate or 4-phenyl butyric acid (PBA), methylamines, and tauroursodeoxycholic acid (TUDCA). Chemical chaperones may be used to influence the protein folding in a cell. Chemical chaperones have been shown in certain instances to correct folding/trafficking defects seen in such diseases as cystic fibrosis (Fischer et al. Am. J. Physiol. Lung Cell Mol. Physiol. 281:L52-L57, 2001), prion-associated diseases, nephrogenic diabetes insipidus, and cancer (Bai et al. Journal of Pharmacological and Toxicological Methods 40(1):39-45, July 1998). Chemical chaperones also find use in the reduction of ER stress and may be useful in the treatment of hypercholesterolemia, atherosclerosis and associated diseases.

“Effective amount”: In general, the “effective amount” of an active agent refers to the amount necessary to elicit the desired biological response. As will be appreciated by those of ordinary skill in this art, the effective amount of an agent may vary depending on such factors as the desired biological endpoint, the agent being delivered, the disease being treated, the subject being treated, etc. For example, the effective amount of agent used to treat or prevent hypercholesterolemia or atherosclerosis is the amount that results in a reduction in blood cholesterol levels by at least about 10%, 20%, 30%, 40%, or 50%.

“Hypercholesterolemia” refers to the presence of high or excessive levels of cholesterol in the blood. Hypercholesterolemia can lead to the development of atherosclerotic plaques in arteries and, eventually, to atherosclerosis. As used herein, the term “hypercholesterolemia” refers to fasting total cholesterol levels above 200 mg/dL.

“Peptide” or “protein”: According to the present invention, a “peptide” or “protein” comprises a string of at least three amino acids linked together by peptide bonds. The terms “protein” and “peptide” may be used interchangeably. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g. greater half-life in vivo). These modifications may include cyclization of the peptide, the incorporation of D-amino acids, etc. None of the modifications should substantially interfere with the desired biological activity of the peptide.

The term “obtaining” as in “obtaining a compound of formula I, formula II or formula III” refers to purchasing, synthesizing or otherwise procuring the compound.

“Polynucleotide” or “oligonucleotide” refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

### BRIEF DESCRIPTION OF THE DRAWINGS

FIG. 1 shows the blood cholesterol levels (mg/dl) of a hypercholesterolemic mouse treated with phosphate buffered saline (PBS) as control and 500 mg/kg of TUDCA.

FIGS. 2a-2c show the results of a hypercholesterolemic mouse study. FIG. 2(a) shows the percent lesion area per full aorta area of a hypercholesterolemic mouse treated with PBS (vehicle control), 50 mg/kg TUDCA (TUD), 500 mg/kg TUDCA, or 10 mg/kg PBA. FIG. 2(b) shows an HPLC analysis of serum of a hypercholesterolemic mouse treated with PBS (vehicle control), 50 mg/kg TUDCA (TUD), 500 mg/kg TUDCA, or 10 mg/kg PBA. Peaks corresponding to VLDL, LDL, and HDL are indicated. FIG. 2(c) shows the blood cholesterol levels (mg/dl) of a hypercholesterolemic mouse treated with vehicle control (PBS); 50 mg/kg TUDCA (TUD), 500 mg/kg TUDCA, or 10 mg/kg PBA. *** indicates p<0.001. (n=3-4 per group)

### DETAILED DESCRIPTION OF CERTAIN PREFERRED EMBODIMENTS OF THE INVENTION

The invention provides agents/compounds or pharmaceutical compositions that can be used to treat or prevent atherosclerosis, stroke, and other ischemic vascular diseases, dyslipidemia and hypercholesterolemia and prevent complications of these conditions. The administration of an effective dose of such an agent, or a combination therapy including such an agent, to a subject to treat or prevent treat atherosclerosis, stroke, and other ischemic vascular diseases, dyslipidemia and hypercholesterolemia and prevent complications of these conditions, may cure the disease being treated, alleviate or reduce at least one sign or symptom of the disease being treated, reduce the short term consequences of the disease, reduce the long term consequences of the disease, or provide some other transient beneficial effect to the subject.

In certain embodiments, the inventive treatment decreases blood cholesterol levels. In other embodiments, the inventive treatment prevents the long term consequences of hypercholesterolemia including atherosclerosis, stroke, ischemic vascular diseases, and dyslipidemia.

In certain embodiments, the inventive treatment may reduce levels of ER stress markers (e.g., spliced forms of XBP-1, phosphorylation status of PERK, phosphorylation of eIF2α, mRNA levels of GRP78/BIP, protein levels of GRP78/BIP, JNK activity) in cells (e.g., adipocytes, hepatocytes).

In certain embodiments, combinations of one or more chemical chaperones may be used. In certain embodiments, the agent is administered in divided doses (e.g., twice per day, three times a day, four times a day, five times a day). In other embodiments, the agent is administered in a single dose per day.

The agent may be combined with one or more other pharmaceutical agents, particularly agents traditionally used in the treatment of hypercholesterolemia and/or atherosclerosis. A list of agents useful in combination with compounds of the invention (e.g., PBA, TUDCA, TMAO, or derivatives thereof) is included as Table 1. The list includes generic names, trade names, and manufacturers. Exemplary agents useful in combination with compounds of the invention include, but are not limited to, anti-diabetic agents (e.g., insulin, hypoglycemic agents (e.g., oral hypoglycemic agents such as sulfonylureas, tolbutamide, metformin, chlorpropamide, acetohexamide, tolazamide, glyburide, etc.)), anti-obesity agents, anti-dyslipidemia agent or anti-atherosclerosis agent (e.g., cholesterol lowering agents (e.g., HMg-CoA reductase inhibitors such as lovastatin, atorvastatin, simvastatin, pravastatin, fluvastatin, etc., aspirin), anti-obesity agent (e.g., appetite suppressants), vitamins, minerals, and anti-hypertensive agents.

In certain embodiments, compounds of the invention (e.g., PBA, TUDCA, TMAO, or derivatives thereof) are used in combination with an anti-dyslipidemia agent or anti-atherosclerosis agent. Exemplary anti-dyslipidemia agents or anti-atherosclerosis agents include HMG-CoA reductase inhibitors (e.g. atorvastatin, pravastatin, simvastatin, lovastatin, fluvastatin, cerivastatina, rosuvastatin, pitivastatin), fibrates (e.g., ciprofibrate, bezafibrate, clofibrate, fenofibrate, gemfibrozil), bile acid sequestrants (e.g., cholestyramine, colestipol, colesevelam), niacin (immediate and extended release), anti-platelet agents (e.g., aspirin, clopidogrel, ticlopidine), angiotensin-converting enzyme (ACE) inhibitors (e.g., ramipril, enalapril), angiotensin II receptor antagonists (e.g., losartan potassium), acyl-CoA cholesterol acetyltransferase (ACAT) inhibitors (e.g.,avasimibe, eflucimibe, CS-505 (Sankyo and Kyoto), SMP-797 (Sumito)), cholesterol absorption inhibitors (e.g., ezetimibe, pamaqueside), nicotinic acid derivatives (e.g., nicotinic acid), cholesterol ester transfer protein (CETP) inhibitors (e.g., CP-529414 (Pfizer), JTT-705 (Japan Tobacco), CETi-1, torcetrapib), microsomal triglyceride transfer protein (MTTP) inhibitors (e.g., implitapide, R-103757, CP-346086 (Pfizer)), other cholesterol modulators (e.g., NO-1886 (Otsuka/TAP Pharmaceutical), CI-1027 (Pfizer), WAY-135433 (Wyeth-Ayerst)), bile acid modulators (e.g., GT102-279 (GelTex/Sankyo), HBS-107 (Hisamitsu/Banyu), BTG-511 (British Technology Group), BARI-1453 (Aventis), S-8921 (Shionogi), SD-5613 (Pfizer), AZD-7806 (AstraZeneca)), peroxisome proliferation activated receptor (PPAR) agonists (e.g., Tesaglitazar (AZ-242) (AstraZeneca), Netoglitazone (MCC-555) (Mitsubishi/Johnson & Johnson), GW-409544 (Ligand Pharmaceuticals/GlaxoSmithKline), GW-501516 (Ligand Pharmaceuticals/GlaxoSmithKline), LY-929 (Ligand Pharmaceuticals and Eli Lilly), LY-465608 (Ligand Pharmaceuticals and Eli Lilly), LY-518674 (Ligand Pharmaceuticals and Eli Lilly), MK-767 (Merck and Kyorin)), gene-based therapies (e.g., AdGVVEGF121.10 (GenVec), ApoA1 (ICB Pharma/Groupe Fournier), EG-004 (Trinam) (Ark Therapeutics), ATP-binding cassette transporter-A1 (ABCA1) (CV Therapeutics/Incyte, Aventis, Xenon)), composite vascular protectant (e.g. AGI-1067 (Atberogenics)), BO-653 (Chugai), glycoprotein IIb/IIIa inhibitors (e.g., Roxifiban (Bristol-Myers Squibb), Gantofiban (Yamanouchi), Cromafiban (Millennium Pharmaceuticals)), aspirin and analogs thereof (e.g., asacard, slow-release aspirin, pamicogrel), combination therapies (e.g., niacin/lovastatin, amlodipine/atorvastatin, simvastatin/ezetimibe), IBAT inhibitors (e.g., S-89-21 (Shionogi)), squalene synthase inhibitors (e.g., BMS-188494 I(Bristol-Myers Squibb), CP-210172 (Pfizer), CP-295697 (Pfizer), CP-294838 (Pfizer), TAK-475 (Takeda)), monocyte chemoattractant protein (MCP-1) inhibitors (e.g., RS-504393 (Roche Bioscience), other MCP-1 inhibitors (GlaxoSmithKline, Teijin, and Bristol-Myers Squibb)), liver X receptor agonists (e.g., GW-3965 (GlaxoSmithKline), TU-0901317 (Tularik)), and other new approaches (e.g., MBX-102 (Metabolex), NO-1886 (Otsuka), Gemcabene (Pfizer)).

In still other embodiments, compounds of the invention (e.g., PBA, TUDCA, TMAO, or derivatives thereof) are used in combination with an anti-hypertensive agent. Examplary anti-hypertension agents include diurectics (e.g., chlorthalidone, metolazone, indapamide, bumetanide, ethacrynic acid, furosemide, torsemide, amiloride HCl, spironolactone, triamterene), alpha-blockers (e.g., doxazosin mesylate, prazosin HCl, terazosin HCl), beta-blockers (e.g., acebutolol, atenolol, betaxolol, bisoprolol fumarate, carteolol HCl, metoprolol tartrate, metoprolol succinate, nadolol, penbutolol sulfate, pindolol, propanolol HCl, timolol maleate, carvedilol), Ca⁺² channel blockers (e.g., amlodipine besylate, felodipine, isradipine, nicardipine, nifedipine, nisoldipine, diltiazem HCl, verapamil HCl, azelnidipine, pranidipine, graded diltiazem formulation, (s)-amlodipine, clevidipine), angiotensin converting enzyme (ACE) inhibitors (e.g., benazepril hydrochloride, captopril, enalapril maleate, fosinopril sodium, lisinopril, moexipril, perindopril, quinapril hydrochloride, ramipril, trandolapril), angiotensin II (AT-II) antagonists (e.g., losartan, valsartan, irbesartan, candesartan, telmisartan, eprosartan, olmesarta, YM-358 (Yamanouchi)), vasopeptidase inhibitors (e.g. omapatrilat, gemopatrilat, fasidotril, sampatrilat, AVE 7688 (Aventis), M100240 (Aventis), Z13752A (Zambon/GSK), 796406 (Zambon/GSK)), dual neutral endopeptidase and enotheline converting enzyme (NEP/ECE) inhibitors (e.g. SLV306 (Solvay), NEP inhibitors (e.g., ecadotril), aldosterone antagonists (e.g., eplerenone), renin inhibitors (e.g., Aliskiren (Novartis), SPP 500 (Roche/Speedel), SPP600 (Speedel), SPP 800 (Locus/Speedel)), angiotensin vaccines (e.g., PMD-3117 (Protherics)), ACE/NEP inhibitors (e.g., AVE-7688 (Aventis), GW-660511 (Zambon SpA)), Na⁺/K⁺ ATPase modulators (e.g., PST-2238 (Prassis-Sigma-Tau), endothelin antagonists (e.g., PD-156707 (Pfizer)), vasodilators (e.g., NCX-4016 (NicOx), LP-805 (Pola/Wyeth)), naturetic peptides (e.g., BDNP (Mayo Foundation)), angiotensin receptor blockers (ARBs) (e.g., pratosartan), ACE crosslink breakers (e.g., alagebrium chloride), endothelin receptor agonists (e.g., tezosentan (Genentech), ambrisentan (Myogen), BMS 193884 (BMS), sitaxsentan (Encysive Pharmaceuticals), SPP301 (Roche/Speedel), Darusentan (Myogen/Abbott), J104132 (Banyu/Merck & Co.), TBC3711 (Encysive Pharmaceuticals), SB 234551 (GSK/Shionogi)), combination therapies (e.g., benazepril hydrochloride/hydrochlorothiazide, captopril/hydrochlorothiazide, enalapril maleate/hydrochlorothiazide, lisinopril/hydrochlorothiazide, losartan/hydrochlorothiazide, atenolol/chlorthalidone, bisoprolol fumarate/hydrochlorothiazide, metoprolol tartrate/hydrochlorothiazide, amlodipine besylate/benazepril hydrochloride, felodipine/enalapril maleate, verapamil hydrochloride/trandolapril, lercanidipine and enalapril, olmesartan/hydrochlorothiazide, eprosartan/hydrochlorothiazide, amlodipine besylate/atorvastatin, nitrendipine/enalapril), and MC4232 (University of Manitoba/Medicure).

In certain embodiments, compounds of the invention (e.g. PBA, TUDCA, TMAO, or derivatives thereof) are used in combination with a vitamin, mineral, or other nutritional supplement.

In certain embodiments, compounds of the invention (e.g., PBA, TUDCA, TMAO, or derivatives thereof) are administered in a sub-optimal dose (e.g. an amount that does not manifest detectable therapeutic benefits when administered in the absence of a second agent). In such cases, the administration of such an sub-optimal dose of a compound of the invention in combination with another agent results in a synergistic effect. The compound of the invention and other agent work together to produce a therapeutic benefit. In other embodiments, the other agent (i.e., not the compound of the invention) is administered in sub-optimal doses. In combination with a compound of the invention, the combination exhibits a therapeutic effect. In yet other embodiments, both the compound of the invention and the other agent are administered in sub-therapeutic doses, and when combined produce a therapeutic effect. The dosages of the other agent may be below those standardly used in the art.

The dosages, route of administration, formulation, etc. for anti-diabetic agents, anti-obesity agents, anti-dyslipidemia agent or anti-atherosclerosis agent, anti-obesity agent, vitamins, minerals, and anti-hypertensive agents (listed above) are known in the art. The treating physician or health care professional may consult such references as the Physician 's Desk Reference (59^{th} Ed., 2005), or Mosby's Drug Consult and Interactions (2005) for such information. It is understood that a treating physician would exercise professional judgment to determine the dosage regimen for a particular patient.

The invention provides systems and methods of treating hypercholesterolemia, atherosclerosis and other related conditions that provide a better therapeutic profile than the administration of a compound of the invention or the other treatment modality alone. In certain embodiments, the therapeutic effect may be greater. In certain embodiments, the combination has a synergistic effect. In other embodiments, the combination has an additive effect. The administration of a combination treatment regimen may reduce or even avoid certain unwanted or adverse side effects. In certain embodiments, the agents in the combination may be administered in lower doses, administered less frequently, or administered less frequently and in lower doses. Therefore, combination therapies with the above described benefits may increase patient compliance, improve therapeutic outcomes, and/or reduce unwanted or adverse side effects.

In certain embodiments, small molecule compounds according to the invention include 4-phenyl butyrate (PBA), tauroursodeoxycholic acid (TUDCA), and trimethylamine N-oxide (TMAQ). PBA is used currently to treat α1-anti-trypsin deficiency, urea cycle disorders, and cystic fibrosis. Derivatives, salts (e.g. sodium, magnesium, potassium, magnesium, ammonium, etc.), prodrugs, esters, isomers, and stereoisomers of PBA, TUDCA, or TMAO may also be used to treat hypercholesterolemia, atherosclerosis and related diseases. Without wishing to be bound by any particular theory, these compounds are thought to work by allowing the ER to better handle misfolded and/or mutant proteins being processed by the ER.

In certain embodiments, a derivative of 4-phenyl butyrate useful in the present invention is of the formula:

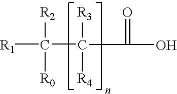

wherein n is 1 or 2;
R₀ is aryl, heteroaryl, or phenoxy, wherein the aryl, heteroaryl, and phenoxy being unsubstituted or substituted with, independently, one or more halogen, hydroxy, or lower alkyl (C₁-C₆) groups;
R₁ and R₂ are independently H, lower alkoxy, hydroxy, lower alkyl or halogen; and
R₃ and R₄ are independently H, lower alkyl, lower alkoxy or halogen; or
a pharmaceutically-acceptable salt thereof; or a mixture thereof. In certain embodiments, R₀ is a substituted or unsubstituted phenyl ring. In certain embodiments, R₀ is an unsubstituted phenyl ring. In other embodiments, R₀ is a monosubstituted phenyl ring. In yet other embodiments, R₀ is a disubstituted phenyl ring. In still other embodiments, R₀ is a trisubstituted phenyl ring. In certain embodiments, R₀ is a phenyl ring substituted with 1, 2, 3, or 4 halogen atoms. In certain embodiments, R₀ is a substituted or unsubstituted heteroaryl ring. In certain embodiments, R₀ is a naphthyl ring. In certain embodiments, R₀ is five- or six-membered, preferably six-membered. In certain embodiments, R₁ and R₂ are both hydrogen. In certain embodiments, n is 1. In other embodiments, n is 2. In certain embodiments, all R₃ and R₄ are hydrogen. In other embodiments, at least one R₃ or R₄ is hydrogen. In certain embodiments, the compound is used in a salt form (e.g. sodium salt, potassium salt, magnesium salt, ammonium salt, etc.) Other derivatives useful in the present invention are described in U.S. Pat. No. 5,710,178. 4-phenyl butyrate or its derivatives may be obtained from commercial sources, or prepared by total synthesis or semi-synthesis.

In certain embodiments, the compound is PBA.

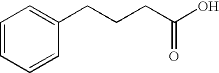

In certain embodiments, a derivative of TUDCA useful in the present invention is of the formula:

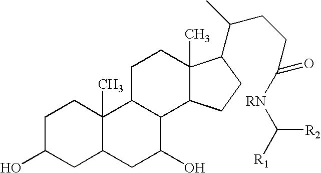

wherein:
R is —H or C₁-C₄ alkyl;

R₁ is —CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or a basic amino acid; or a pharmaceutically acceptable salt thereof. In certain embodiments, the stereochemistry of the derivative is defined as shown in the following structure:

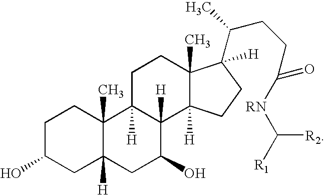

In certain embodiments, R is H. In other embodiments, R is methyl, ethyl, n-propyl, iso-propyl, n-butyl, iso-butyl, or tert-butyl, preferably, methyl. In certain embodiments, R₁ or R₂ is hydrogen. In certain embodiments, R₁ is —CH₂—SO₃R₃ and R₂ is —H. In other embodiments, R₁ is —COOH and R₂ is —CH₂—CH₂—CONH₂, —CH₂—CONH₂, —CH₂—CH₂—SCH₃ or —CH₂—S—CH₂—COOH. In certain embodiments, R₃ is hydrogen. In certain embodiments, R3 is lysine, arginine, ornithine, or histidine. Derivatives of TUDCA and ursodeoxycholic acid may be obtained from commercial sources, prepared from total synthesis, or obtained from a semi-synthesis. In certain embodiments, the derivative is prepared via semi-synthesis, for example, as described in U.S. Pat. Nos. 5,550,421 and 4,865,765.

In certain embodiments, derivative of trimethylamine N-oxide useful in the present invention is of the formula:

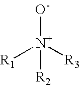

wherein
R₁, R₂, and R₃ are independently hydrogen, halogen, or lower C₁-C₆ alkyl; or

a pharmaceutically-acceptable salt thereof; or a mixture thereof. In certain embodiments, R₁, R₂, and R₃ are the same. In other embodiments, at least one of R₁, R₂, and R₃ is different. In yet other embodiments, all of R₁, R₂, and R₃ are different. In certain embodiments, R₁, R₂, and R₃ are independently hydrogen or lower C₁-C₆ alkyl. In yet other embodiments, R₁, R₂, and R₃ are independently lower C₁-C₆ alkyl. In still other embodiments, R₁, R₂, and R₃ are independently methyl, ethyl, or propyl. In certain embodiments, R₁, R₂, and R₃ are ethyl. Derivatives of TMAO may be obtained from commercial sources, or prepared by total synthesis or semi-synthesis.

In certain embodiments, the methods of the invention further comprise obtaining the compounds of formula I, II and/or III. In other embodiments, the methods of the invention further comprise identifying a subject as being in need of in prevention or treatment of hypercholesterolemia and/or atherosclerosis.

In accordance with certain embodiments of the invention, a therapeutically effective amount of a compound of the invention is administered to the subject via any route to achieve the desired biological result. Any route of administration may be used including orally, parenterally, intravenously, intraarterially, intramuscularly, subcutaneously, rectally, vaginally, transdermally, intraperitoneally, and intrathecally. In certain embodiments, the compound is administered parenterally. In other embodiments, the compound is administered orally. In the use of PBA, TUDCA, or TMAO, the compound is preferably administered orally; however, any of the administration routes listed above may also be used. In certain embodiments, the PBA, TUDCA, or TMAO is administered parenterally. In certain embodiments, the comound is administered in divided doses (e.g., twice per day, three times a day, four times a day, five times a day). In other embodiments, the compound is administered in a single dose per day.

Pharmaceutical Compositions

Pharmaceutical compositions of the present invention and for use in accordance with the present invention may include a pharmaceutically acceptable excipient or carrier. As such, the compositions of the present invention can be used for the formulation of a medicament in combination with a pharmaceutically acceptable carrier. As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), transdermally, subcutaneously, bucally, or as an oral or nasal spray.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

The pharmaceutical compositions of the invention may be provided in a kit with other agents used to treat or prevent hypercholesterolemia and/or atherosclerosis. The kit may include instructions for the treating physician and/or patient, which may include dosing information, safety information, list of side effects, chemical formula of agent, mechanism of action, etc. In certain embodiments, the kit may include materials for administering the pharmaceutical composition. For example, the kit may include a syringe, needle, alcohol swabs, etc. for the administration of an injectable preparation. In certain embodiments when two or more agents are provided in a kit, the active pharmaceutical ingredients may be formulated separately or together. For example, the kit may include a first container with a compound of the invention (e.g., PBA, TUDCA, TMAO, or a derivative thereof) and a second container with a second agent used in treating hypercholesterolemia and related diseases. In certain embodiments, the active pharmaceutical ingredients are formulated separately. In other embodiments, the active pharmaceutical ingredients are formulated together.

### Exemplification

### Example 1

### Effect of TUCA and PBA on Hypercholesterolemic Mice

This example demonstrates that TUDCA is effective: (1) in alleviating atherosclerotic lesions on aortic tissue isolated from hypercholesterolemic mice; and (2) in lowering blood cholesterol levels in blood samples obtained from hypercholesterolemic mice.

apoE−/− mice (a commonly used model of hypercholesterolemia and atherosclerosis) were purchased from Jackson Labs. At 6 weeks of age, the mice were placed on a western diet (to further increase cholesterol levels and vascular lesions) and treatments with either phosphate buffered saline (PBS) (vehicle) or TUDCA (500 mg/kg/day, single dose, i.p.) were started.

After 3 months of western diet and treatment, mice were sacrificed and aortas were dissected and fixed in 10% buffered formalin and stained with Oil-Red-O which stains lipids. Aortas from PBS treated animals had substantially more Oil-Red-O staining than the mice treated with TUDCA, demonstrating that TUDCA is useful in the prevention and treatment of the formation of atherosclerotic plaques.

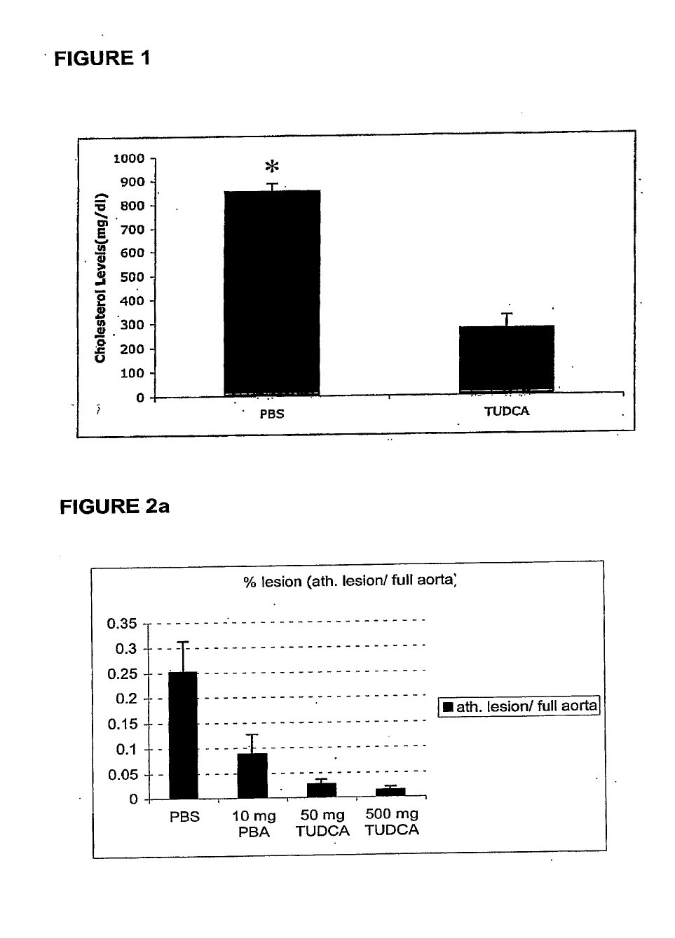

Blood samples were obtained from the animals described above and serum cholesterol levels were determined by the Piccolo automated device with lipid panel (Abaxis, Calif.). FIG. 1 shows that TUDCA treatment significantly (p<0.001) lowered cholesterol levels in the animals described above. These results demonstrate that TUDCA is useful in the lowering of serum cholesterol and the prevention and treatment of hypercholesterolemia.

PBA was tested under similar conditions and a dose of about 10 mg/kg/day was found to be effective in reducing the amount of atherosclerotic lesion present relative to control treated animals.

### Example 2

### Effect of TUCA and PBA on Hypercholesterolemic Mice

This example demonstrates that both TUDCA and PBA are effective: (1) in alleviating atherosclerotic lesions on aortic tissue isolated from hypercholesterolemic mice; and (2) that the decrease in atherosclerosis is not dependent upon a decrease in total cholesterol or VLDL levels. The example further demonstrates that at a dose of 500 mg/kg TUDCA is effective: (1) in lowering total blood cholesterol levels in blood samples obtained from hypercholesterolemic mice; and (2) decreasing VLDL and LDL levels, while increasing HDL levels.

apoE−/− mice were purchased from Jackson Labs. At 6 weeks of age, the mice were placed on a western diet and treatments with either phosphate buffered saline (PBS) (vehicle); TUDCA (50 mg/kg/day or 500 mg/kg/day, single dose, i.p.); or 4-PBA (10 mg/kg/day, 200 mg/kg/day, or 1 g/kg/day, single dose, oral gavage) were started.

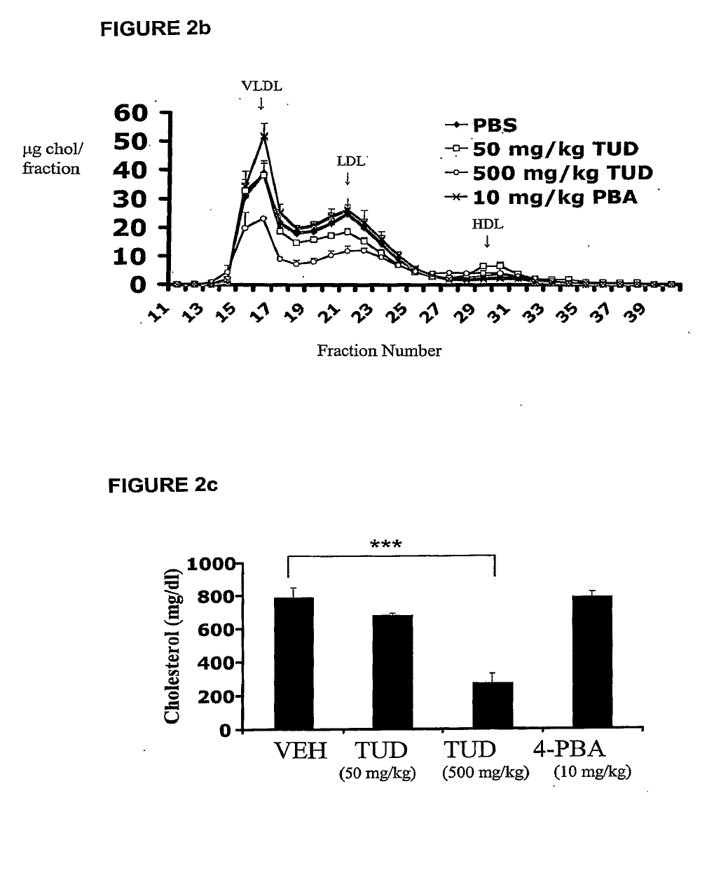

After 3 months of western diet and treatment, mice were sacrificed and aortas were dissected and fixed in 10% buffered formalin and stained with Oil-Red-O. The amount of atherosclerotic lesion area per full aorta was determine and is expressed as a percent as shown in FIG. 2(a). Mice receiving vehicle (PBS) developed robust atherosclerotic lesions in the aortic arch as well as in the thoracic and abdominal parts of the aorta (0.25%±0.06). Administration of TUDCA at 50 mg/kg/day or 500 mg/kg/day completely blocked the development of atherosclerosis in the animals (0.03%±0.01 and 0.01%±0.01, respectively, n=3 per group, p<0.05 vs. control). PBA administration at a dose of 10 mg/kg/day was also found to be effective in reducing formation of atherosclerotic lesions (0.09%±0.03, n=4, p<0.05 vs. control). However, at the higher doses no similar reduction was observed.

Blood samples were obtained from the animals described above. Lipoprotein profiles analysis was performed by HPLC (FIG. 2b), and total cholesterol levels (FIG. 2c). Total lipoprotein analysis revealed a significant decrease in total cholesterol, VLDL, and LDL; and a marked increase in HDL, in response to treatment with 500 mg/kg/day of TUDCA. Surprisingly, no significant changes in total cholesterol or specific lipoprotein levels were observed in response to treatment with either 50 mg/kg/day of TUDCA or 4-PBA, despite the difference in the amount of atherosclerotic lesion present. These data demonstrate that the antiatheroscleorotic effects of a compound can be separable from the cholesterol lowering effects of the compound. Moreover, the ability to reduce the development of atherosclerotic lesions is not dependent on the ability of a compound to reduce cholesterol.

TABLE I
Drugs for Used in Combination with Compounds of the Invention

ANTI-ATHEROSCLEROSIS DRUGS
HMG-CoA reductase inhibitors (statins)
Atorvastatin (Warner-Lambert/Pfizer's Lipitor)
Pravastatin (Bristol-Myers Squibb's Pravachol/Sankyo's Mevalotin)
Simvastatin (Merck & Co.'s Zocor)
Lovastatin (Merck & Co.'s Mevacor)
Fluvastatin (Novartis's Lescol)
Cerivastatina (Bayer's Lipobay/GlaxoSmithKline's Baycol)
Rosuvastatin (AstraZeneca's Crestor)
Pitivastatin (itavastatin/risivastatin) (Nissan/Kowa/Sankyo/Novartis)
Fibrates
Bezafibrate (Boehringer Mannheim/Roche's Bezalip, Kissei's Bezatol)
Clofibrate (Wyeth-Ayerst's Atromid-S, generics)
Fenofibrate (Fournier's Lipidil, Abbott's Tricor, Takeda's Lipantil, generics)
Gemfibrozil (Pfizer's Lopid, generics)
Bile acid sequestrants
Cholestyramine Bristol-Myers Squibb's Questran and Questran Light, generics
Colestipol Pharmacia's Colestid
Niacin
Niacin-immediate release (Aventis's Nicobid, Upsher-Smith's Niacor,
Aventis's Nicolar, Sanwakagaku's Perycit, generics
Niacin-extended release (Kos Pharmaceuticals' Niaspan, Upsher-Smith's Slo-
Niacin)
Antiplatelet agents
Aspirin (Bayer's Aspirin, generics)
Clopidogrel (Sanofi-Synthélabo/Bristol-Myers Squibb's Plavix)
Ticlopidine (Sanofi-Synthélabo's Ticlid, Daiichi's Panaldine, generics)
Angiotensin-converting enzyme inhibitors
Ramipril (Aventis's Altace)
Enalapril (Merck & Co.'s Vasotec)
Angiotensin II receptor antagonists
Losartan potassium (Merck & Co.'s Cozaar)
Acyl CoA cholesterol acetyltransferase (ACAT) inhibitors
Avasimibe (Pfizer)
Eflucimibe (BioMérieux Pierre Fabre/Eli Lilly)
CS-505 (Sankyo and Kyoto)
SMP-797 (Sumito)
Cholesterol absorption inhibitors
Ezetimibe (Schering-Plough/Merck & Co.)
Pamaqueside (Pfizer)
Cholesterol ester transfer protein (CETP) inhibitors
CP-529414 (Pfizer)
JTT-705 (Japan Tobacco)
CETi-1 (Avant Immunotherapeutics)
Microsomal triglyceride transfer protein (MTTP) inhibitors
Implitapide (Bayer)
R-103757 (Janssen)
Other cholesterol modulators
NO-1886 (Otsuka/TAP Pharmaceutical)
CI-1027 (Pfizer)
WAY-135433 (Wyeth-Ayerst)
Bile acid modulators
GT102-279 (GelTex/Sankyo)
HBS-107 (Hisamitsu/Banyu)
Peroxisome proliferation activated receptor (PPAR) agonists
Tesaglitazar (AZ-242) (AstraZeneca)
Netoglitazone (MCC-555) (Mitsubishi/Johnson & Johnson)
GW-409544 (Ligand Pharmaceuticals/GlaxoSmithKline)
GW-501516 (Ligand Pharmaceuticals/GlaxoSmithKline)
Gene-based therapies
AdGVVEGF121.10 (GenVec)
ApoA1 (UCB Pharma/Groupe Fournier)
EG-004 (Trinam) (Ark Therapeutics)
ATP-binding cassette transporter-A1 (ABCA1) (CV Therapeutics/Incyte,
Aventis, Xenon)
Composite vascular protectants
AGI-1067 (Atherogenics)
Other anti-atherosclerotic agents
BO-653 (Chugai Pharmaceuticals)
Glycoprotein IIb/IIIa inhibitors
Roxifiban (Bristol-Myers Squibb)
Gantofiban (Yamanouchi)
Cromafiban (Millennium Pharmaceuticals)
Aspirin and aspirin-like compounds
Asacard (slow-release aspirin) (Pharmacia)
Pamicogrel (Kanebo/Angelini Ricerche/CEPA)
Combination therapies
Advicor (niacin/lovastatin) (Kos Pharmaceuticals)
Amlodipine/atorvastatin (Pfizer)
Simvastatin/ezetimibe (Merck & Co./Schering-Plough)
IBAT inhibitors
S-8921 (Shionogi)
Squalene synthase inhibitors
BMS-188494 I(Bristol-Myers Squibb)
CP-210172 (Pfizer)
CP-295697 (Pfizer)
CP-294838 (Pfizer)
Monocyte chemoattractant protein (MCP)-1 inhibitors
RS-504393 (Roche Bioscience)
Other MCP-1 inhibitors (GlaxoSmithKline, Teijin, and Bristol-Myers Squibb)
Other Drugs from the PDR:
Indication = Hypercholesterolemia
Advicor Tablets (Kos)
Lovastatin, Niacin
Altoprev Extended-Release Tablets (Andrx Labs)
Lovastatin
Caduet Tablets (Pfizer)
Amlodipine Besylate, Atorvastatin Calcium
Crestor Tablets (AstraZeneca)
Rosuvastatin Calcium
Lescol Capsules (Novartis)
Fluvastatin Sodium
Lescol Capsules (Reliant)
Fluvastatin Sodium
Lescol XL Tablets (Novartis)
Fluvastatin Sodium
Lescol XL Tablets (Reliant)
Fluvastatin Sodium
Lipitor Tablets (Parke-Davis)
Atorvastatin Calcium
Lofibra Capsules (Gate)
Fenofibrate
Mevacor Tablets (Merck)
Lovastatin
Niaspan Extended-Release Tablets (Kos)
Niacin
Pravachol Tablets (Bristol-Myers Squibb)
Pravastatin Sodium
Tricor Tablets (Abbott)
Fenofibrate
Vytorin 10/10 Tablets (Merck/Schering Plough)
Ezetimibe, Simvastatin
Vytorin 10/10 Tablets (Schering)
Ezetimibe, Simvastatin
Vytorin 10/20 Tablets (Merck/Schering Plough)
Ezetimibe, Simvastatin
Vytorin 10/20 Tablets (Schering)
Ezetimibe, Simvastatin
Vytorin 10/40 Tablets (Merck/Schering Plough)
Ezetimibe, Simvastatin
Vytorin 10/40 Tablets (Schering)
Ezetimibe, Simvastatin
Vytorin 10/80 Tablets (Merck/Schering Plough)
Ezetimibe, Simvastatin
Vytorin 10/80 Tablets (Schering)
Ezetimibe, Simvastatin
WelChol Tablets (Sankyo)
Colesevelam Hydrochloride
Zetia Tablets (Schering)
Ezetimibe
Zetia Tablets (Merck/Schering Plough)
Ezetimibe
Zocor Tablets (Merck)
Simvastatin
ANTI- DYSLIPIDEMIA DRUGS
HMG-CoA reductase inhibitors
Atorvastatin (Pfizer's Lipitor/Tahor/Sortis/Torvast/Cardyl)
Simvastatin (Merck's Zocor/Sinvacor, Boehringer Ingelheim's Denan, Banyu's
Lipovas)
Pravastatin (Bristol-Myers Squibb's Pravachol, Sankyo's Mevalotin/Sanaprav)
Fluvastatin (Novartis's Lescol/Locol/Lochol, Fujisawa's Cranoc, Solvay's
Digaril)
Lovastatin (Merck's Mevacor/Mevinacor, Bexal's Lovastatina, Cepa; Schwarz
Pharma's Liposcler)
Rosuvastatin (AstraZeneca's Crestor)
Pitavastatin (Nissan Chemical, Kowa Kogyo, Sankyo, and Novartis)
HMG-CoA reductase inhibitor combination therapies
Simvastatin/ezetimibe (Merck and Schering-Plough)
Fibrates
Fenofibrate (Abbott's Tricor, Fournier's Lipidil/Lipantil)
Bezafibrate (Roche's Béfizal/Cedur/Bezalip, Kissei's Bezatol, generics)
Gemfibrozil (Pfizer's Lopid/Lipur, generics)
Clofibrate (Wyeth's Atromid-S, generics)
Ciprofibrate (Sanofi-Synthelabo's Modalim)
Bile acid sequestrants
Colestyramine (Bristol-Myers Squibb's Questran)
Colestipol (Pfizer's Colestid)
Colesevelam (Genzyme/Sankyo's Welchol)
Cholesterol absorption inhibitors
Ezetimibe (Merck and Schering-Plough's Zetia)
Pamaqueside (Pfizer)
Nicotinic acid derivatives
Nicotinic acid (Kos's Niaspan, Yamanouchi's Nyclin)
Acyl-CoA cholesterol acyltransferase inhibitors
Avasimibe (Pfizer)
Eflucimibe (Eli Lilly)
Cholesteryl ester transfer protein inhibitors
Torcetrapib (Pfizer)
JTT-705 (Japan Tobacco)
CETi-1 (Avant Immunotherapeutics)
Microsomal triglyceride transfer protein inhibitors
Implitapide (Bayer)
CP-346086 (Pfizer)
Peroxisome proliferation activated receptor agonists
GW-501516 (Ligand Pharmaceuticals and GlaxoSmithKline)
Tesaglitazar (AstraZeneca)
LY-929 (Ligand Pharmaceuticals and Eli Lilly)
LY-465608 (Ligand Pharmaceuticals and Eli Lilly)
LY-518674 (Ligand Pharmaceuticals and Eli Lilly)
MK-767 (Merck and Kyorin)
Squalene synthase inhibitors
TAK-475 (Takeda)
Other new approaches
MBX-102 (Metabolex)
NO-1886 (Otsuka)
Gemcabene (Pfizer)
Liver X receptor agonists
GW-3965 (GlaxoSmithKline)
TU-0901317 (Tularik)
Bile acid modulators
BTG-511 (British Technology Group)
HBS-107 (Hisamitsu and Banyu)
BARI-1453 (Aventis)
S-8921 (Shionogi)
SD-5613 (Pfizer)
AZD-7806 (AstraZeneca)
Other Drugs from the PDR:
Indication = Hypercholesterolemia
Advicor Tablets (Kos)
Lovastatin, Niacin
Altoprev Extended-Release Tablets (Andrx Labs)
Lovastatin
Caduet Tablets (Pfizer)
Amlodipine Besylate, Atorvastatin Calcium
Crestor Tablets (AstraZeneca)
Rosuvastatin Calcium
Lescol Capsules (Novartis)
Fluvastatin Sodium
Lescol Capsules (Reliant)
Fluvastatin Sodium
Lescol XL Tablets (Novartis)
Fluvastatin Sodium
Lescol XL Tablets (Reliant)
Fluvastatin Sodium
Lipitor Tablets (Parke-Davis)
Atorvastatin Calcium
Lofibra Capsules (Gate)
Fenofibrate
Mevacor Tablets (Merck)
Lovastatin
Niaspan Extended-Release Tablets (Kos)
Niacin
Pravachol Tablets (Bristol-Myers Squibb)
Pravastatin Sodium
Tricor Tablets (Abbott)
Fenofibrate
Vytorin 10/10 Tablets (Merck/Schering Plough)
Ezetimibe, Simvastatin
Vytorin 10/10 Tablets (Schering)
Ezetimibe, Simvastatin
Vytorin 10/20 Tablets (Merck/Schering Plough)
Ezetimibe, Simvastatin
Vytorin 10/20 Tablets (Schering)
Ezetimibe, Simvastatin
Vytorin 10/40 Tablets (Merck/Schering Plough)
Ezetimibe, Simvastatin
Vytorin 10/40 Tablets (Schering)
Ezetimibe, Simvastatin
Vytorin 10/80 Tablets (Merck/Schering Plough)
Ezetimibe, Simvastatin
Vytorin 10/80 Tablets (Schering)
Ezetimibe, Simvastatin
WelChol Tablets (Sankyo)
Colesevelam Hydrochloride
Zetia Tablets (Schering)
Ezetimibe
Zetia Tablets (Merck/Schering Plough)
Ezetimibe
Zocor Tablets (Merck)
Simvastatin

### Incorporation by Reference

The contents of all references (including literature references, issued patents, published patent applications, and co-pending patent applications) cited throughout this application are hereby expressly incorporated herein in their entireties by reference.

### Equivalents

Those skilled in the art will recognize, or be able to ascertain using no more than routine experimentation, many equivalents of the specific embodiments of the invention described herein. Such equivalents are intended to be encompassed by the following claims.

1. A method of treating or preventing hypercholesterolemia and/or atherosclerosis in a subject comprising administering to a subject in need thereof a therapeutically effective amount of a TUDCA compound of formula I, 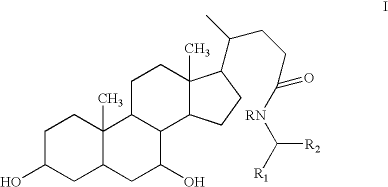
wherein R is —H or C₁-C₄ alkyl;
R₁ is —CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or the residue of a basic amino acid, or
a pharmaceutically acceptable salt or derivative thereof; or a mixture thereof, thereby treating or preventing hypercholesterolemia and/or atherosclerosis in said subject.

2. The method of claim 1, wherein in the compound of formula I, R₁ is —CH₂—SO₃H and R₂ is —H.

3. The method of claim 2, wherein R is —H.

4. The method of claim 1, wherein the compound of formula I is tauroursodeoxycholic acid (TUDCA)

5. The method of claim 1, wherein the compound of formula I is administered at a dose ranging from about 10 mg/kg/day to about 500 mg/kg/day.

6. The method of claim 1, wherein the compound of formula I is administered at a dose of about 50 mg/kg/day.

7. The method of claim 1, wherein the compound of formula I is administered at a dose of about less than 10 mg/kg/day.

8. The method of claim 1, wherein the compound is a derivative, salt, or isomer of TUDCA.

9. A method of treating or preventing hypercholesterolemia and/or atherosclerosis in a subject comprising administering to a subject in need thereof a therapeutically effective amount of a PBA compound of formula II, 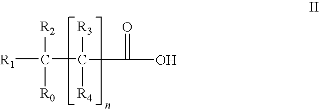
wherein n is 1 or 2;
R₀ is aryl, heteroaryl, or phenoxy, the aryl and phenoxy being unsubstituted or substituted with, independently, one or more halogen, hydroxy or lower alkyl;
R₁ and R₂ are independently H, lower alkoxy, hydroxy, lower alkyl or halogen; and
a pharmaceutically-acceptable salt thereof; or a mixture thereof, thereby treating or preventing hypercholesterolemia and/or atherosclerosis in said subject.

10. The method of claim 9, wherein in the compound of formula II,
R₀ is phenyl, naphthyl, or phenoxy, the phenyl, naphthyl and phenoxy being unsubstituted or substituted with, independently, one or more moieties of halogen, hydroxy or lower alkyl.

11. The method of claim 9, wherein in the compound of formula II,
R₀ is phenyl, naphthyl, or phenoxy, the phenyl, naphthyl and phenoxy being unsubstituted or substituted with, independently, from 1 to 4 moieties of halogen, hydroxy or lower alkyl of from 1 to 4 carbon atoms;
R₁ and R₂ are, independently, H, hydroxy, lower alkoxy of from 1 to 2 carbon atoms, lower straight or branched chain alkyl of from 1 to 4 carbon atoms or halogen; and
R₃ and R₄ are, independently, H, lower alkoxy of from 1 to 2 carbon atoms, lower straight or branched chain alkyl of from 1 to 4 carbon atoms or halogen.

12. The method of claim 9, wherein in the compound of formula II, n is 1.

13. The method of claim 9, wherein in the compound of formula II, n is 2.

14. The method of claim 9, wherein in the compound of formula II, R₀ is phenyl.

15. The method of claim 9, wherein in the compound of formula II, R₀ is substituted phenyl.

16. The method of claim 9, wherein in the compound of formula II, the substitution on the phenyl at R₀ is from 1 to 4 halogen moieties.

17. The method of claim 9, wherein in the compound of formula II, R₃ and R₄ are both —H.

18. The method of claim 9, wherein the compound of formula II is 4-phenyl butyric acid (PBA)

19. The method of claim 9, wherein the compound of formula II is administered at a dose ranging from about 1 mg/kg/day to about less 1000 mg/kg/day.

20. The method of claim 9, wherein the compound of formula II is administered at a dose of about 200 mg/kg/day.

21. The method of claim 9, wherein the compound of formula II is administered at a dose of about 10 mg/kg/day.

22. The method of claim 9, wherein the compound is a derivative, salt, or isomer of PBA.

23. A method of treating or preventing hypercholesterolemia and/or atherosclerosis in a subject comprising administering to a subject in need thereof a therapeutically effective amount of a TMAO compound of formula III, 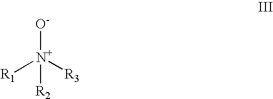
wherein
R₁, R₂, and R₃ are independently hydrogen, halogen, or lower C₁-C₆ alkyl; or
a pharmaceutically-acceptable salt thereof; or a mixture thereof, thereby treating or preventing hypercholesterolemia and/or atherosclerosis in said subject.

24. The method of claim 23, wherein in the compound of formula III, R₁, R₂, and R₃ are independently lower C₁-C₆ alkyl.

25. The method of claim 23, wherein the compound of formula III is trimethylamine N-oxide (TMAO).

26. The method of claim 23, wherein the compound is a derivative, salt or isomer of TMAO.

27. The method of claim 1, wherein the subject is a mammal.

28. The method of claim 1, wherein the subject is a human.

29. The method of claim 1, wherein the step of administering comprises administering the compound orally.

30. The method of claim 1, wherein the step of administering comprises administering the compound parenterally.

31. The method of claim 30, wherein the step of administering comprises administering the compound intravenously.

32. The method of claim 1, further comprising identifying a subject as being in need of prevention or treatment of hypercholesterolemia and/or atherosclerosis.

33. The method of claim 1, further comprising obtaining the compound of formula I.

34. The method of claim 1, comprising further administering a compound selected from the group consisting of an antiatherosclerotic drug, an antidyslipidemia drug, and a drug indicated for hypercholesterolemia.

35. A pharmaceutical composition comprising a therapeutically effective amount for treating or preventing hypercholesterolemia and/or atherosclerosis of a compound as recited in claim 1 and a pharmaceutically acceptable diluent or carrier.

36. A pharmaceutical composition comprising a therapeutically effective amount for treating or preventing hypercholesterolemia and/or atherosclerosis of a compound as recited in claim 9 and a pharmaceutically acceptable diluent or carrier.

37. A pharmaceutical composition comprising a therapeutically effective amount for treating or preventing hypercholesterolemia and/or atherosclerosis of a compound as recited in claim 23 and a pharmaceutically acceptable diluent or carrier.

38. A kit comprising a compound as recited in claim 1 and instructions directing the use of said compound for treating or preventing hypercholesterolemia and/or atherosclerosis in accordance with the method of claim 1.

39. A kit comprising a compound as recited in claim 9 and instructions directing the use of said compound for treating or preventing hypercholesterolemia and/or atherosclerosis accordance with the method of claim 9.
